(19) United States
(12) Patent Application Publication
(10) Pub. No.: US 2026/0080653 A1
(43) Pub. Date: Mar. 19, 2026

(54) IMAGE PROCESING METHOD, IMAGE PROCESSING DEVICE, AND PROGRAM

(71) Applicant: National University Corporation Chiba University, Chiba-shi, Chiba (JP)

(72) Inventors: Hideyuki MIYAUCHI, Chiba-shi, Chiba (JP); Takashi IIMORI, Chiba-shi, Chiba (JP); Koichi SAWADA, Chiba-shi, Chiba (JP)

(21) Appl. No.: 19/109,046

(22) PCT Filed: Sep. 5, 2023
(86) PCT No.: PCT/JP2023/032407

§ 371 Date: Mar. 5, 2025

(30) Foreign Application Priority Data
Sep. 6, 2022 (JP) 2022-141634

(51) Int. Cl. G06V 10/25; G01T 1/164; G16H 30/20

(52) U.S. Cl. CPC G06V 10/25; G01T 1/164; G16H 30/20

(57) ABSTRACT

The present invention is an image processing method for calculating the washout rate (WR) (%) of a radiopharmaceutical in a region of interest (ROI) of a subject to which the radiopharmaceutical has been administered, the method including a step of calculating the washout rate (%) by Formula (I). (Formula (I) is as defined in Description.)

### TECHNICAL FIELD

The present invention relates to an image processing method, an image processing device, and a program. In particular, the image processing method relates to an image processing method for providing the washout rate (%) in a region of interest in a subject to which a radiopharmaceutical has been administered.

### BACKGROUND ART

The washout rate (WR) of a radiopharmaceutical is used in, for example, evaluating the clinical condition of a heart using myocardial nuclear imaging. In diagnosis of triglyceride deposit cardiomyovasculopathy (TGCV), measurement of the washout rate using myocardial scintigraphy with ¹²³I-BMIPP (¹²³I-β-methyl-p-iodophenyl-pentadecanoic acid) representative as a radiopharmaceutical is one of the essential criteria for diagnosing TGCV.

Alternatively, as radiopharmaceuticals, thallium chloride (²⁰¹Tl), ¹²³I-MIBG (¹²³I-meta-iodobenzylguanidine) and the like are used to measure the washout rate.

As described above, correct calculation of the washout rate is significantly important for clinical condition identification and diagnoses of cardiovascular diseases.

NPL 1 describes measurement of the washout rate of 201Tl from cardiac muscle.

### CITATION LIST

### Non Patent Literature

NPL 1: Bateman T M, et. al. J Am Coll Cardiol. 1984 Jul. 4 (1) 55-64.

### SUMMARY OF INVENTION

### Technical Problem

The present inventors have found that a conventional washout rate calculating method cannot correctly reflect the true washout rate, and requires improvement of the calculation method.

The present invention relates to an image processing method, an image processing device, and a program that can more correctly calculate the washout rate.

### Solution to Problem

The present inventors have found that the problem described above can be solved by the following aspects <1> to <10>.

<1> An image processing method for calculating a washout rate (WR) (%) of a radiopharmaceutical in a region of interest (ROI) of a subject to which the radiopharmaceutical has been administered,
the image processing method sequentially performing the following steps, the image processing method being used together with a method of creating an image by reconstructing data obtained by performing single-photon emission computed tomography (SPECT) imaging on a subject and converting the data into three-dimensional information, subsequently segmenting the obtained three-dimensional information into a freely selected number of three-dimensional-information-retaining segments and determining representative count values of the respective segments, and thereafter converting the representative count values of the segments into a visually-comparable image:
(1) a step of setting a region of interest in each of a first image created based on data obtained by imaging the subject to which the radiopharmaceutical has been administered, and a second image created based on data obtained by imaging the subject at a time after obtaining the first image,
(2) a step of identifying the three-dimensional-information-retaining segments included in each region of interest, and obtaining the representative count values of all the three-dimensional-information-retaining segments included in the corresponding region of interest, and
(3) a step of calculating the washout rate (%) by the following Formula (I),

### Formula (I):

$$washout rate ( \% ) = \{ ( sum of representative count values of all three - dimensional - information - retaining segments included in the region of interest in the first image ) - ( sum of representative count values of all three - dimensional - information - \text{} retaining segments included in the region of interest in the second image ) \} / ( sum of representative count values of all three - dimensional - information - retaining segments included in the region of interest in the first image ) \times 100.$$

<2> The image processing method according to <1>, wherein the image is an image where the three-dimensional-information-retaining segments are displayed according to polar coordinate system.

<3> The image processing method according to <1> or <2>, wherein the three-dimensional information is created with a filtered back projection method (FBP method) or an ordered subset expectation maximization method (OSEM method).

<4> The image processing method according to any one of <1> to <3>, wherein the region of interest is set to a heart.

<5> The image processing method according to any one of <1> to <4>, wherein the radiopharmaceutical contains one or more types of radionuclides selected from a group consisting of radioactive thallium, radioactive technetium, and radioactive iodine.

<6> The image processing method according to any one of <1> to <5>, wherein the subject is a patient with decreased uptake of ¹²³I-BMIPP into a heart, and is a subject affected with or suspected to have triglyceride deposit cardiomyovasculopathy (TGCV).

<7> The image processing method according to <6>, wherein a factor of the decreased uptake of ¹²³I-BMIPP into the heart is old myocardial infarction (OMI).

<8> The image processing method according to any one of <1> to <7>, wherein the washout rate (%) to be used to diagnose as triglyceride deposit cardiomyovasculopathy (TGCV) is provided.

<9> An image processing device, including a data obtainer and a data processor,
wherein the data obtainer is an obtainer of data obtained by performing single-photon emission computed tomography (SPECT) imaging on a subject to which a radiopharmaceutical has been administered,
the data processor includes:
(0) means for creating an image by reconstructing SPECT imaging data obtained by the data obtainer and converting the data into three-dimensional information, subsequently segmenting the obtained three-dimensional information into a freely selected number of three-dimensional-information-retaining segments and determining representative count values of the respective segments, and thereafter converting the representative count values of the segments into a visually-comparable image;
(1) means for setting a region of interest (ROI) in each of a first image created based on data obtained by imaging the subject to which the radiopharmaceutical has been administered, and a second image created based on data obtained by imaging the subject at a time after obtaining the first image;
(2) means for identifying the three-dimensional-information-retaining segments included in each region of interest, and obtaining the representative count values of all the three-dimensional-information-retaining segments included in the corresponding region of interest; and
(3) means for calculating the washout rate (%) by the following Formula (I),

### Formula (I):

$$washout rate ( \% ) = \{ ( sum of representative count values of all three - dimensional - information - retaining segments included in the region of interest in the first image ) - ( sum of representative count values of all three - dimensional - information - \text{} retaining segments included in the region of interest in the second image ) \} / ( sum of representative count values of all three - dimensional - information - retaining segments included in the region of interest in the first image ) \times 100.$$

<10> A program for causing a computer to execute each step of the image processing method according to any one of <1> to <8>.

### Advantageous Effects of Invention

According to the present invention, an image processing method, an image processing device, and a program that can more correctly calculate the washout rate are provided.

### BRIEF DESCRIPTION OF DRAWINGS

FIG. 1 schematically shows the distributions of counts in a region of interest (ROI); (a) indicates a case of the present invention, and (b) indicates a case of a conventional method.

FIG. 2 schematically shows the distributions of counts in the region of interest (ROI); (a) indicates a case of the present invention, and (b) indicates a case of a conventional method.

FIG. 3 schematically shows the distributions of counts in the region of interest (ROI); (a) indicates a case of the present invention, and (b) indicates a case of a conventional method.

FIG. 4 shows calculation results of washout rates.

FIG. 5 shows representative polar coordinate system images.

### DESCRIPTION OF EMBODIMENTS

### [Image Processing Method]

An image processing method in the present invention is an image processing method for calculating a washout rate (WR) (%) of a radiopharmaceutical in a region of interest (ROI) of a subject to which the radiopharmaceutical has been administered, the image processing method being used together with a method of creating an image by reconstructing data obtained by performing single-photon emission computed tomography (SPECT) imaging on a subject and converting the data into three-dimensional information, subsequently segmenting the obtained three-dimensional information into a freely selected number of three-dimensional-information-retaining segments and determining representative count values of the respective segments, and thereafter converting the representative count values of the segments into a visually-comparable image.

The image processing method in the present invention sequentially performs the following steps:
(1) a step of setting a region of interest in each of a first image created based on data obtained by imaging the subject to which the radiopharmaceutical has been administered, and a second image created based on data obtained by imaging the subject at a time after obtaining the first image,
(2) a step of identifying the three-dimensional-information-retaining segments included in each region of interest, and obtaining the representative count values of all the three-dimensional-information-retaining segments included in the corresponding region of interest, and
(3) a step of calculating the washout rate (%) by the following Formula (I),

### Formula (I):

$$washout rate ( \% ) = \{ ( sum of representative count values of all three - dimensional - information - retaining segments included in the region of interest in the first image ) - ( sum of representative count values of all three - dimensional - information - \text{} retaining segments included in the region of interest in the second image ) \} / ( sum of representative count values of all three - dimensional - information - retaining segments included in the region of interest in the first image ) \times 100.$$

### <Washout Rate>

The image processing method in the present invention is a method for providing the washout rate (%) of a radiopharmaceutical in a region of interest (ROI) of a subject to which the radiopharmaceutical has been administered.

The washout rate (WR) relates to a temporal distribution of the radiopharmaceutical in the region of interest, and is a numerical value obtained by quantifying a phenomenon in which the radiopharmaceutical is accumulated in the region of interest initially and is washed out over time.

The washout rate is provided as a piece of information on the region of interest of the subject. One preferable aspect of the present invention is a myocardial fatty acid metabolism scintigraphy that involves the administration of ¹²³I-BMIPP as a radiopharmaceutical, and employs the heart as the region of interest, and can diagnose as triglyceride deposit cardiomyovasculopathy based on the washout rate, for example.

The washout rate (%) is calculated by the following Formula (I).

### Formula (I):

$$washout rate ( \% ) = \{ ( sum of representative count values of all three - dimensional - information - retaining segments included in the region of interest in the first image ) - ( sum of representative count values of all three - dimensional - information - \text{} retaining segments included in the region of interest in the second image ) \} / ( sum of representative count values of all three - dimensional - information - retaining segments included in the region of interest in the first image ) \times 100.$$

### <Subject>

Test subjects of the image processing method in the present invention are not specifically limited as long as subjects for whom the washout rate is required to be measured, and include, for example, mammals including humans. Non-human mammals include monkeys, mice, rats, rabbits, dogs, cats, cattle, sheep, horses, pigs and the like.

In a case where test subjects are humans, the gender, race, and chronological age are not limited.

In one preferable aspect of the present invention, subjects are subjects who undergo myocardial scintigraphy scanning. For example, for myocardial fatty acid metabolism scintigraphy, subjects are patients with decreased uptake of ¹²³I-BMIPP into their hearts. More specifically, for example, they are subjects affected with or suspected to have triglyceride deposit cardiomyovasculopathy (TGCV).

For example, patients having necrotic myocardium have decreased uptake of ¹²³I-BMIPP into their hearts. These include a case where the factor of decreased uptake of ¹²³I-BMIPP into the heart is old myocardial infarction (OMI).

### <Region of Interest>

The image processing method in the present invention can set regions of interest (ROIs) to various types of organs and tissue in a body. For example, regions of interest can be set to a heart, a brain, a liver, muscles, kidneys, a pancreas, and tumor lesions. It is preferable to set the region of interest to a heart, or a brain among them in view of the capability to diagnose severe diseases.

The region of interest may be one region, or a combination of two or more regions. The region may also be the entirety of or part of a specific organ.

For the myocardial fatty acid metabolism scintigraphy, which is one of the preferable aspects of the present invention, the entirety of or part of the heart is employed as a region of interest.

### <Radiopharmaceutical>

The radiopharmaceutical that can be used in the present invention is not specifically limited. For example, a radiopharmaceutical that contains one or more types of radionuclides selected from a group consisting of radioactive thallium, radioactive technetium, and radioactive iodine can be selected according to the purpose.

More specifically, a radiopharmaceutical that contains radioactive thallium may be thallium chloride (²⁰¹Tl) etc.

A radiopharmaceutical that contains radioactive technetium may be technetium (^{99m}Tc) tetrofosmin, hexakis (2-methoxyisobutylisonitrile) technetium (^{99m}Tc) (^{99m}Tc-MIBI), etc.

A radiopharmaceutical that contains radioactive iodine may be ¹²³I-meta-iodobenzylguanidine (¹²³I-MIBG), ¹²³I-β-methyl-p-iodophenyl-pentadecanoic acid (¹²³I-BMIPP), etc.

### <Image Created by SPECT>

The image processing method in the present invention is used together with a method of creating an image by reconstructing data obtained by performing SPECT imaging on a subject and converting the data into three-dimensional information, subsequently segmenting the obtained three-dimensional information into a freely selected number of three-dimensional-information-retaining segments and determining representative count values of the respective segments, and thereafter converting the representative count values of the segments into a visually-comparable image. That is, the image processing method in the present invention is executed in relation to obtainment of an image created by SPECT.

The image created by SPECT can be performed by analysis software that the SPECT device includes.

The image created by SPECT is provided as a diagnostic image that visually represents the radiopharmaceutical distribution, by, for example, display output, printer output or the like. Preferably, a region-of-interest portion is extracted, and provided.

According to the purpose, the region of interest is displayed in a divided manner. For example, Heart Risk View-S (HRV-S), which is SPECT analysis software made by Nihon Medi-Physics Co., Ltd., can divide an image created by SPECT into 0 divisions (entirety), 3 divisions, 5 divisions, 17 divisions, and 20 divisions. Preferably, the number of divisions is 0 (entirety) or 17.

The image processing method in the present invention can be performed at the same time as obtainment of an image created by SPECT or immediately after the obtainment. Based on an existing obtained image created by SPECT, the method can be performed at a later time on the same day or on a later date. As the existing obtained image created by SPECT, for example, what is recorded in a computer-readable medium can be used.

For imaging by SPECT, a SPECT device that includes a gamma camera is used, gamma rays emitted from the radiopharmaceutical are detected, and a count value is measured. Measurement data is reconstructed and converted into three-dimensional information, subsequently the obtained three-dimensional information is segmented into a freely selected number of three-dimensional-information-retaining segments, representative count values of the respective segments are determined, subsequently the representative count values of the segments are converted into a visually-comparable image, and an image is created, thus allowing the radiopharmaceutical distribution to be visually identified as an image.

The image created by SPECT is an image created from three-dimensional information reconstructed based on two-dimensional information obtained from a three-dimensional subject in a plurality of directions. Accordingly, unlike a planar image obtained by one-directional imaging, an image can be created from which radiopharmaceutical distributions in other organs, tissue and the like which three-dimensionally overlap with the region of interest are excluded.

A specific method of reconstructing measurement data and converting the data into three-dimensional information may be a publicly known filtered back projection method (FBP method), ordered subset expectation maximization method (OSEM method), etc.

The three-dimensional information includes position information and a count value.

### <Three-Dimensional-Information-Retaining Segment>

In data processing for obtaining an image created by SPECT, three-dimensional information is segmented into a freely selected number of three-dimensional-information-retaining segments. The number of three-dimensional-information-retaining segments is not specifically limited as long as obtained information can be processed in a state suited to the purpose. For example, it is preferable that the number of segments range from 500 to 10,000, more preferably, range from 1,000 to 5,000. As specific examples, about 1,500 segments, 2,048 segments (16×128), and 2,400 segments (20×120) can be exemplified. However, there is no limitation to them. As examples of segmenting into a freely selected number of segments, SPECT analysis software products provided by various manufacturers can be exemplified.

Hereinafter, an aspect is described where Heart Risk View-S (HRV-S), i.e., SPECT analysis software made by Nihon Medi-Physics Co., Ltd., segments a heart region into a freely selected number of three-dimensional-information-retaining segments.

First, the heart region is divided into toric shapes having different sizes perpendicularly to the long axis center line. Note that the cardiac apex is subjected to another special process. More specifically, the number of divisions in the long axis direction is 20.

Next, each toric shape portion is subjected to circumferential profile analysis of division by radially drawing lines from the long axis center line. More specifically, for example, 120 divisions with units of 3 degrees, or 60 divisions with units of 6 degrees can be achieved.

In the case of circumferential profile analysis with 120 divisions, the heart region is divided into 2,400 segments.

Note that the division into three-dimensional-information-retaining segments is division into segments that are typically different from divided representation of the region of interest according to the purpose.

Next, as to the obtained three-dimensional-information-retaining segments, representative count values are determined in the respective segments. Each segment consists of a plurality of volumes of interest (VOIs). The number of incident gamma rays emitted from the radiopharmaceutical per unit time period in each VOI is obtained as a count value by the SPECT device.

The representative count value in each segment is selected based on the count values of a plurality of VOIs belonging to the segment concerned. For example, the maximum, mode, median, minimum, mean or the like of the count values of the plurality of VOIs belonging to the segment concerned can be adopted as a representative count value of this segment. Preferably, the representative count value is the maximum.

Next, the representative values of the respective segments are converted so as to be visually comparable, and a visually identifiable image is created. For example, the representative count value is applied to a color scale or a grayscale, colors corresponding to the respective count values (shades in the grayscale) are determined, and an image is generated.

The generated image is obtained as, for example, a polar map according to polar coordinate system.

Creation of the image may be performed by analysis software that the SPECT device includes. Alternatively, information on any stage (e.g., reconstructed three-dimensional information) may be transferred to another computer, and the creation may be performed by dedicated software.

### <Step (1)>

The image processing method in the present invention includes, as step (1), setting a region of interest in each of a first image created based on data obtained by imaging the subject to which the radiopharmaceutical has been administered, and a second image created based on data obtained by imaging the subject at a time after obtaining the first image.

### (First Image and Second Image)

The first image created based on data obtained by imaging the subject to which the radiopharmaceutical has been administered, and the second image created based on data obtained by imaging the subject at a time after obtaining the first image are obtained by the image generating means described above.

The first image is also called an early image, and is obtained by imaging a predetermined time period after administration of the radiopharmaceutical to the subject. The predetermined time period after the administration can be set appropriately, depending on the type of the radiopharmaceutical. For example, in myocardial fatty acid metabolism scintigraphy where ¹²³I-BMIPP is administered, it is preferable that the predetermined time period after administration be 5 to 60 minutes after the administration of the radiopharmaceutical, more preferably, 10 to 40 minutes after the administration, and further preferably, 15 to 30 minutes after the administration.

The second image is also called a delayed image, and is obtained by imaging the subject at a time after the first imaging. The time after the first imaging can be set appropriately, depending on the type of the radiopharmaceutical. For example, in the myocardial fatty acid metabolism scintigraphy where ¹²³I-BMIPP is administered, it is preferable that the period be 1 to 6 hours after the first imaging, more preferably, 2 to 5 hours after the first imaging, and further preferably, 3 to 4 hours after the first imaging.

### (Setting of Region of Interest (ROI))

In each of the first image and the second image, a region of interest (ROI) is set.

The region of interest can be set so as to include a freely selected number of three-dimensional-information-retaining segments. The region of interest can be set by, for example, the analysis software in the SPECT device according to a publicly known method.

A lapse of time from first SPECT imaging to second SPECT imaging reduces the radioactivity of the radiopharmaceutical. Accordingly, it is preferable that the count value obtained by the second SPECT imaging be subsequently corrected to an attenuation-corrected value. The attenuation correction can be performed based on the radiopharmaceutical to be used, and on the time interval from the first SPECT imaging to the second SPECT imaging.

### <Step (2)>

Next, in step (2), the three-dimensional-information-retaining segments included in each region of interest are identified, and the representative count values of all the three-dimensional-information-retaining segments included in the corresponding region of interest are obtained.

According to step (2), in each of the region of interest in the first image and the region of interest in the second image, the representative count values associated with the respective three-dimensional-information-retaining segments are obtained.

### <Step (3)>

Next, in step (3), the washout rate is calculated by the following Formula (I).

### Formula (I):

$$washout rate ( \% ) = \{ ( sum of representative count values of all three - dimensional - information - retaining segments included in the region of interest in the first image ) - ( sum of representative count values of all three - dimensional - information - \text{} retaining segments included in the region of interest in the second image ) \} / ( sum of representative count values of all three - dimensional - information - retaining segments included in the region of interest in the first image ) \times 100.$$

Data obtained in step (2) is used as the representative count values of the three-dimensional-information-retaining segments included in the region of interest in the first image, and the representative count values of the three-dimensional-information-retaining segments included in the region of interest in the second image.

According to the image processing method in the present invention, even in a case where the radiopharmaceutical is unevenly distributed, the washout rate can be more correctly calculated. The mechanism where the present invention exerts advantageous effects is described below.

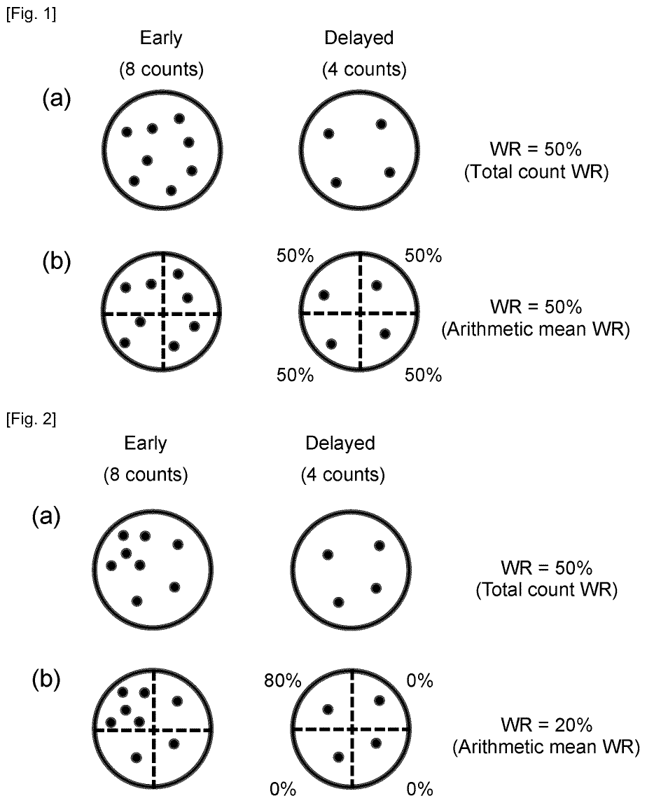

Hereinafter, the method of the present invention and the conventional method are compared with each other with reference to FIGS. 1 to 3. FIGS. 1 to 3 schematically show the radiopharmaceutical distribution in the region of interest (ROI). In the drawings, solid black circles indicate presence of the radiopharmaceutical. In the drawings, “Early” indicates early images corresponding to the first image, and “Delayed” indicates delayed images corresponding to the second image. Note that (a) and (b) in FIGS. 1 to 3 represent the same region of interest.

In FIGS. 1 to 3, (a) indicates the case of the present invention. In the present invention that calculates the washout rate by Formula (I), all the representative count values included in the region of interest are added together. Accordingly, the boundaries of the three-dimensional-information-retaining segments are not indicated in the drawings. In the present description, the method of calculating the washout rate by Formula (I) in the present invention is also called a total count method.

In the conventional method, the washout rate was calculated by the following Formula (II).

### Formula (II):

$$WR ( \% ) = \frac{{\sum }_{i = 1}^{n} \frac{{x}_{i} - {y}_{i}}{{x}_{i}} \times 100}{n} \qquad [ Expression 1 ]$$

In the formula, n is an integer representing the number of three-dimensional-information-retaining segments, xᵢ is the representative count value of the i-th three-dimensional-information-retaining segment in the first image, and yᵢ is the representative count value of the i-th three-dimensional-information-retaining segment in the second image.

In each of FIGS. 1 to 3, (b) indicates the case of the conventional method. The conventional method that calculates the washout rate by Formula (II) performs calculation with respect to each three-dimensional-information-retaining segment. Accordingly, the drawings indicate that the region of interest consists of four three-dimensional-information-retaining segments. Hereinafter, in the present description, the washout rate calculating method by the conventional Formula (II) is also called an arithmetic mean method.

In FIG. 1(a), the sum of solid black circles included in the region of interest is eight in the early image, and the sum of solid black circles included in the region of interest is four in the delayed image. In this case, according to the total count method, it is calculated that WR=50% by Formula (I).

In FIG. 1(b), the region of interest consists of four three-dimensional-information-retaining segments. In the early image, two solid black circles are included in each of the upper left segment, the upper right segment, the lower left segment, and the lower right segment. In the delayed image, one solid black circle is included in each of the upper left segment, the upper right segment, the lower left segment, and the lower right segment. In this case, according to the arithmetic mean method, it is calculated that WR=50% by Formula (II).

FIG. 2 shows a case where the radiopharmaceutical is unevenly distributed.

In FIG. 2(a), the sum of solid black circles included in the region of interest is eight in the early image, and the sum of solid black circles included in the region of interest is four in the delayed image. In this case, according to the total count method, it is calculated that WR=50% by Formula (I).

In FIG. 2(b), in the early image, the number of solid black circles included in the upper left segment is five, the number of solid black circles included in the upper right segment is one, the number of solid black circles included in the lower left segment is one, and the number of solid black circles included in the lower right segment is one. In the delayed image, the number of solid black circles included in the upper left segment is one, the number of solid black circles included in the upper right segment is one, the number of solid black circles included in the lower left segment is one, and the number of solid black circles included in the lower right segment is one. In this case, according to the arithmetic mean method, it is calculated that WR=20% by Formula (II).

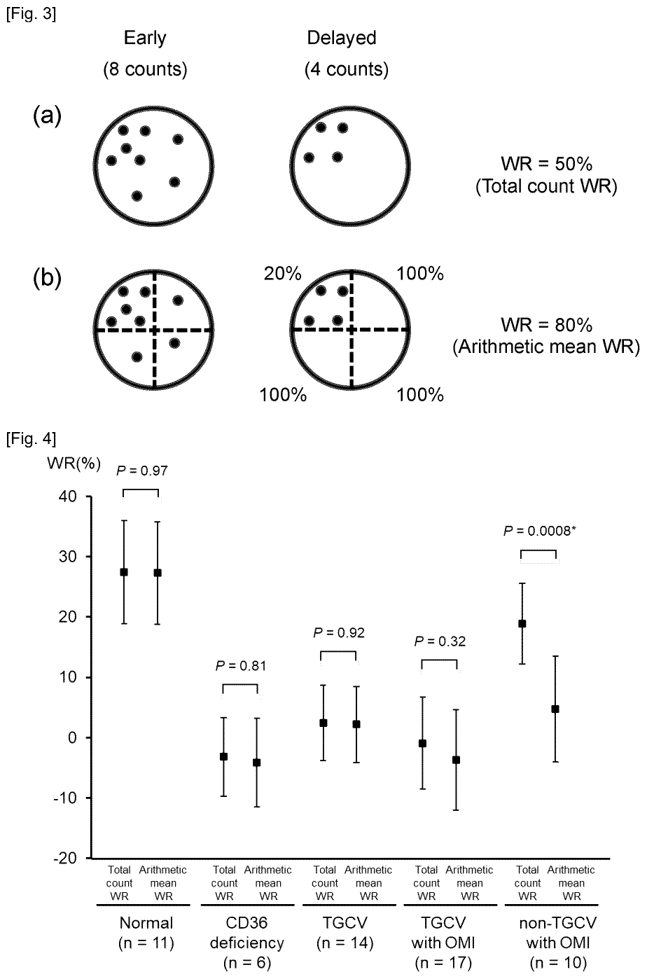

FIG. 3 shows another case where the radiopharmaceutical is unevenly distributed.

In FIG. 3(a), the sum of solid black circles included in the region of interest is eight in the early image, and the sum of solid black circles included in the region of interest is four in the delayed image. In this case, according to the total count method, it is calculated that WR=50% by Formula (I).

In FIG. 3(b), in the early image, the number of solid black circles included in the upper left segment is five, the number of solid black circles included in the upper right segment is one, the number of solid black circles included in the lower left segment is one, and the number of solid black circles included in the lower right segment is one. In the delayed image, the number of solid black circles included in the upper left segment is four, the number of solid black circles included in the upper right segment is zero, the number of solid black circles included in the lower left segment is zero, and the number of solid black circles included in the lower right segment is zero. In this case, according to the arithmetic mean method, it is calculated that WR=80% by Formula (II).

As shown in FIGS. 2 and 3, according to the arithmetic mean method, which is the conventional method, the washout rate may not be calculated correctly in the case where the radiopharmaceutical is unevenly distributed.

### [Image Processing Device]

An image processing device in the present invention is an image processing device that includes a data obtainer and a data processor,
wherein the data obtainer is an obtainer of data obtained by performing single-photon emission computed tomography (SPECT) imaging on a subject to which a radiopharmaceutical has been administered,
the data processor includes:
(0) means for creating an image by reconstructing SPECT imaging data obtained by the data obtainer and converting the data into three-dimensional information, subsequently segmenting the obtained three-dimensional information into a freely selected number of three-dimensional-information-retaining segments and determining representative count values of the respective segments, and thereafter converting the representative count values of the segments into a visually-comparable image;
(1) means for setting a region of interest (ROI) in each of a first image created based on data obtained by imaging the subject to which the radiopharmaceutical has been administered, and a second image created based on data obtained by imaging the subject at a time after obtaining the first image;
(2) means for identifying the three-dimensional-information-retaining segments included in each region of interest, and obtaining the representative count values of all the three-dimensional-information-retaining segments included in the corresponding region of interest; and
(3) means for calculating the washout rate (%) by the following Formula (I),

### Formula (I):

$$washout rate ( \% ) = \{ ( sum of representative count values of all three - dimensional - information - retaining segments included in the region of interest in the first image ) - ( sum of representative count values of all three - dimensional - information - \text{} retaining segments included in the region of interest in the second image ) \} / ( sum of representative count values of all three - dimensional - information - retaining segments included in the region of interest in the first image ) \times 100.$$

Preferably, the image processing device in the present invention is a device that can include data that is part of a SPECT system or can be combined with a SPECT imaging device, and a device that can be included as part of the image processing device, and is configured so as to be capable of executing calculation of the washout rate according to the image processing method in the present invention. Preferable aspects are similar to the image processing method described above.

### (Data Obtainer)

A SPECT system or a SPECT imaging device includes, for example, a plurality of gamma radiation detectors, and is configured such that the gamma radiation detectors can rotate around a subject, and detect, in a plurality of directions, gamma rays emitted from the radiopharmaceutical.

The data obtainer that is included as part of the SPECT system or is coupled to the SPECT imaging device includes means for obtaining data, such as position information and intensity information on detected gamma rays, and means for transmitting the obtained data to the data processor.

### (Data Processor)

The data processor includes: (0) means for creating an image by reconstructing SPECT imaging data obtained by the data obtainer and converting the data into three-dimensional information, subsequently segmenting the obtained three-dimensional information into a freely selected number of three-dimensional-information-retaining segments and determining representative count values of the respective segments, and thereafter converting the representative count values of the segments into a visually-comparable image; (1) means for setting a region of interest (ROI) in each of a first image created based on data obtained by imaging the subject to which the radiopharmaceutical has been administered, and a second image created based on data obtained by imaging the subject at a time after obtaining the first image; (2) means for identifying the three-dimensional-information-retaining segments included in each region of interest, and obtaining the representative count values of all the three-dimensional-information-retaining segments included in the corresponding region of interest; and (3) means for calculating the washout rate (%) by Formula (I) described above.

These means are executed by, for example, a program to be executed by a computer, or by, for example, using a workstation of the SPECT system. The program is recorded in, for example, a computer-readable medium.

Furthermore, the data processing device may include input means, such as a keyboard, a mouse, and a touch panel, and output devices, such as a display and a printer.

### [Program]

A program in the present invention is a program for causing a computer to execute each step of the image processing method described above.

### EXAMPLE

Hereinafter, the present invention is described in more detail in Example.

### (Patient)

The following patients were examined, as subjects, with the approval of Chiba University Ethical Review Board.
Normal subjects without cardiovascular disease (Normal) (n=11)
CD36 deficiency patients (n=6)
Triglyceride deposit cardiomyovasculopathy patients (TGCV) (n=14)
TGCV patients with old myocardial infarction (OMI) (TGCV with OMI) (n=17)
Non-TGCV patients with extensive OMI (non-TGCV with OMI) (n=10)

Note that CD36 deficiency patients have a genetic mutation where the function of the CD36 gene related to uptake of fatty acids, such as ¹²³I-BMIPP, is lost, and reduction in uptake of ¹²³I-BMIPP occurs in the early image.

### (Myocardial Fatty Acid Metabolism Scintigraphy)

According to the protocol recommended by the Japanese Society of Nuclear Cardiology, after fasting for 12 hours or longer, 111 MBq of ¹²³I-BMIPP (Cardiodine made by Nihon Medi-Physics Co., Ltd.) was administered intravenously at rest.

In 20 minutes, an early image (“Early” in the drawing) was obtained. In 210 minutes, a delayed image (“Delayed” in the drawing) was obtained. GE Infinia Hawkeye 4 (made by GE HealthCare Japan Corp.) equipped with an extended low-energy general-purpose collimator as a SPECT device was used.

For the delayed image, attenuation correction was performed with a coefficient of 1/0.5 (interval time period/13.2). Note that “interval time period” is an imaging interval time period between the early image and the delayed image.

Imaging conditions are described as follows.
64×64 matrix
180° step and shoot mode
Sampling angle of 6° 60 seconds/view
Pixel size: 5.89 mm
Slice width: 5.89 mm

The energy window of ¹²³I is set to 159 keV±10% (main) and 130 keV±10% (sub).

Image reconstruction was performed using a ramp filter according to the filtered back projection method. Scatter correction was performed using a 10th order Butterworth filter (cutoff frequency of 0.4 cycle/cm).

### (Calculation of Washout Rate (WR))

Heart Risk View-S (HRV-S made by Nihon Medi-Physics Co., Ltd.) was used as myocardial SPECT analysis software.

The washout rate was calculated according to a total count method and an arithmetic mean method described below.

$$Total count WR ( \% ) = \frac{( {x}_{1} + {x}_{2} + … + {x}_{n} ) - ( {y}_{1} + {y}_{2} + … + {y}_{n} )}{( {x}_{1} + {x}_{2} + … + {x}_{n} )} \times 100 = \frac{{\sum }_{i = 1}^{n} {x}_{i} - {\sum }_{i = 1}^{n} {y}_{i}}{{\sum }_{i = 1}^{n} {x}_{i}} \times 100 \qquad [ Expression 2 ]$$

$$Arithmetic mean WR ( \% ) = \frac{\frac{{x}_{1} - {y}_{1}}{{x}_{1}} \times 100 + \frac{{x}_{2} - {y}_{2}}{{x}_{2}} \times 100 + .. + \frac{{x}_{n} - {y}_{n}}{{x}_{n}} \times 100}{n} = \frac{{\sum }_{i = 1}^{n} \frac{{x}_{i} - {y}_{i}}{{x}_{i}} \times 100}{n}$$

In the formula, n is an integer representing the number of three-dimensional-information-retaining segments, xᵢ is the representative count value of the i-th three-dimensional-information-retaining segment constituting the early image, and yᵢ is the representative count value of the i-th three-dimensional-information-retaining segment constituting the delayed image.

### Results

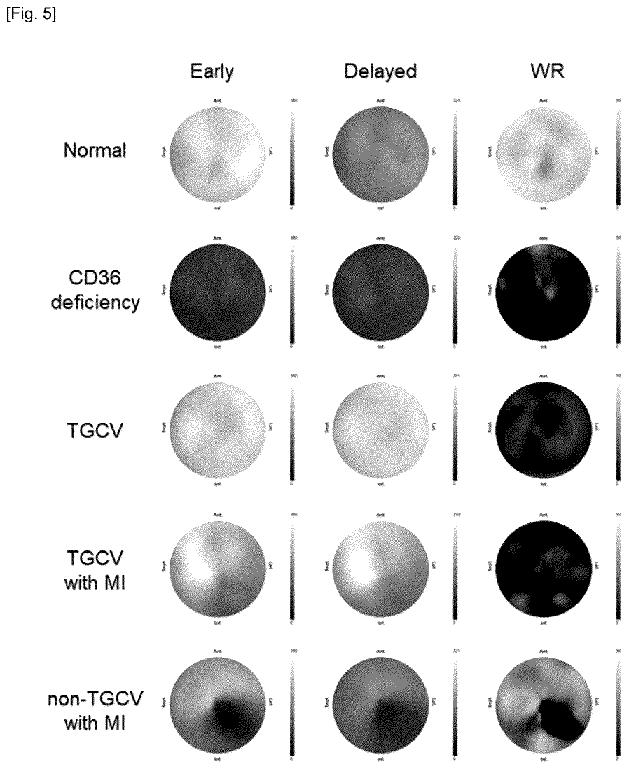

The results are shown in FIG. 4. Representative polar coordinate system images in the corresponding groups are shown in FIG. 5.

Normal subjects, CD36 deficiency patients, triglyceride deposit cardiomyovasculopathy patients, and TGCV patients with OMI showed no significant differences between the washout rates calculated by the total count method and the arithmetic mean method.

However, non-TGCV patients with OMI had a significantly lower washout rate calculated by the arithmetic mean method than that calculated by the total count method.

The low washout rates for the non-TGCV patients with OMI measured by the arithmetic mean method were sometimes lower than the diagnostic criterion of the TGCV. Accordingly, for diagnosis, TGCV is required to be excluded based on other findings.

On the other hand, the total count method in the present invention can correctly calculate the washout rate even for non-TGCV patients with OMI, thus allowing to diagnose as non-TGCV only with the WR value.

1. An image processing method for calculating a washout rate (WR) (%) of a radiopharmaceutical in a region of interest (ROI) of a subject to which the radiopharmaceutical has been administered,
the image processing method sequentially performing the following steps, the image processing method being used together with a method of creating an image by reconstructing data obtained by performing single-photon emission computed tomography (SPECT) imaging on a subject and converting the data into three-dimensional information, subsequently segmenting the obtained three-dimensional information into a freely selected number of three-dimensional-information-retaining segments and determining representative count values of the respective segments, and thereafter converting the representative count values of the segments into a visually-comparable image:
(1) a step of setting a region of interest in each of a first image created based on data obtained by imaging the subject to which the radiopharmaceutical has been administered, and a second image created based on data obtained by imaging the subject at a time after obtaining the first image,
(2) a step of identifying the three-dimensional-information-retaining segments included in each region of interest, and obtaining the representative count values of all the three-dimensional-information-retaining segments included in the corresponding region of interest, and
(3) a step of calculating the washout rate (%) by the following Formula (I),
washout rate (%)={(sum of representative count values of all three-dimensional-information-retaining segments included in the region of interest in the first image)−(sum of representative count values of all three-dimensional-information-retaining segments included in the region of interest in the second image)}/(sum of representative count values of all three-dimensional-information-retaining segments included in the region of interest in the first image)×100. Formula (I):

2. The image processing method according to claim 1, wherein the image is an image where the three-dimensional-information-retaining segments are displayed according to polar coordinate system.

3. The image processing method according to claim 1, wherein the three-dimensional information is created with a filtered back projection method (FBP method) or an ordered subset expectation maximization method (OSEM method).

4. The image processing method according to claim 1, wherein the region of interest is set to a heart.

5. The image processing method according to claim 1, wherein the radiopharmaceutical comprises one or more types of radionuclides selected from a group consisting of radioactive thallium, radioactive technetium, and radioactive iodine.

6. The image processing method according to claim 1, wherein the subject is a patient with decreased uptake of ¹²³I-BMIPP into a heart, and is a subject affected with or suspected to have triglyceride deposit cardiomyovasculopathy (TGCV).

7. The image processing method according to claim 6, wherein a factor of the decreased uptake of ¹²³I-BMIPP into the heart is old myocardial infarction (OMI).

8. The image processing method according to claim 1, wherein the washout rate (%) to be used to diagnose as triglyceride deposit cardiomyovasculopathy (TGCV) is provided.

9. An image processing device, comprising a data obtainer and a data processor,
wherein the data obtainer is an obtainer of data obtained by performing single-photon emission computed tomography (SPECT) imaging on a subject to which a radiopharmaceutical has been administered,
the data processor comprises:
(0) means for creating an image by reconstructing SPECT imaging data obtained by the data obtainer and converting the data into three-dimensional information, subsequently segmenting the obtained three-dimensional information into a freely selected number of three-dimensional-information-retaining segments and determining representative count values of the respective segments, and thereafter converting the representative count values of the segments into a visually-comparable image;
(1) means for setting a region of interest (ROI) in each of a first image created based on data obtained by imaging the subject to which the radiopharmaceutical has been administered, and a second image created based on data obtained by imaging the subject at a time after obtaining the first image;
(2) means for identifying the three-dimensional-information-retaining segments included in each region of interest, and obtaining the representative count values of all the three-dimensional-information-retaining segments included in the corresponding region of interest; and
(3) means for calculating the washout rate (%) by the following Formula (I),
Formula (I):
$$washout rate ( \% ) = \{ ( sum of representative count values of all three - dimensional - information - retaining segments included in the region of interest in the first image ) - ( sum of representative count values of all three - dimensional - information - \text{} retaining segments included in the region of interest in the second image ) \} / ( sum of representative count values of all three - dimensional - information - retaining segments included in the region of interest in the first image ) \times 100.$$

10. A program for causing a computer to execute each step of the image processing method according to claim 1.
